(19) United States
(12) Patent Application Publication
(10) Pub. No.: US 2010/0069788 A1
(43) Pub. Date: Mar. 18, 2010

(54) DEVICE FOR THE SAMPLING OF TISSUES IN BIOPSY PROCEDURES

(76) Inventors: Marcello Dell'Oca, Andalo Valtellino (IT); Lorenzo Dell'Oca, Delebio (IT)

Correspondence Address: HEDMAN & COSTIGAN P.C., 1185 AVENUE OF THE AMERICAS, NEW YORK, NY, 10036, US

(21) Appl. No.: 12/312,952

(22) PCT Filed: Nov. 26, 2007
(86) PCT No.: PCT/IB2007/003755

§ 371 Date: Sep. 10, 2009

(30) Foreign Application Priority Data
Dec. 6, 2006 (IT) M12006A 002360

(51) Int. Cl. A61B 10/02

(52) U.S. Cl. 600/567

(57) ABSTRACT

A device 10 for the sampling of tissues in biopsy procedures comprising a withdrawal group 11 situated inside a cannula 15, wherein said withdrawal group 11 consists of a withdrawal element 12 provided with an end 16 and a sliding element 13 with an end 18 which can be moved from a first position not overlying said end 16 and a second position interposed between said cannula 15 and said end 16.

The present invention relates to the field of medical devices for biopsy procedures, in particular it relates to a device for the sampling of tissues.

Bioptic tissue samples are typically obtained from the patient with the use of sampling devices. These devices comprise a hollow cannula in which a mandrel is inserted. The mandrel has a sharpened tip which protrudes from a distal point, in turn sharpened, of the hollow cannula. The combination of cannula and mandrel is used for penetrating the outer wall of the tissue. Once the cannula and mandrel have reached the tissue, the mandrel is removed and the cannula is advanced allowing the penetration of a part of the medullar tissue inside the cavity of the device.

The sampling is then effected, i.e. the removal of a part of the tissue of interest.

There are currently various kinds of systems for effecting this sampling, which differ from each other.

Among these, the most well-known methods are known as “luxation”, “Trap System” and “Trap matic”, “Safe Cut”, and the “folded flap” method.

In the sampling system by “luxation”, after the tissue had been introduced into the cannula, a side luxation movement of the needle is effected, which, by bending, causes the tissue to become detached in the apical part. This system is the most economical and traditionally the most widely-used.

In the “Trap System” method, the cannula is equipped with a cylindrical tube with the apical part milled in which a cylindrical device, specifically called trap system, upon coming into contact with said apical cone, bends internally and causes a closing of the tip which grips the tissue.

In the “Safe Cut” method a bent iron wire is introduced into the rear part of the cannula and its rotation allows the cutting of the tissue.

Finally, by means of the “folded flap” method, a folded flap positioned on the cannula is inserted like a valve into the fragment detaching it from the underlying tissue.

All the above devices and relative techniques however have disadvantages such as the presence of a luxation in the detachment operation of the tissue, which causes a great trauma for the patient, or the doctor must insert a further device into the instrument during the sampling operation.

Associated with this procedure is also the high risk of damaging the tissue in the use of springs or valves which penetrate it and the surface irregularity of the cannulas currently used, which could cause the insemination of tumoral cells during the extraction of the needle.

Finally, unfortunately, the acquisition of tissues is often effected in insufficient quantities.

An objective of the present invention is to provide a device capable of solving the above-mentioned disadvantages of the known art in an extremely simple, economical and particularly functional manner.

Another objective is to provide a device for the sampling of tissues in biopsy procedures without having to effect painful luxation operations of the gripped tissue.

A further objective is to be able to have a device for the sampling of tissues in biopsy procedures in which it is no longer necessary to insert a further device in the cannula during the sampling operation.

Yet another objective is to provide a device for the sampling of tissues in biopsy procedures which allows a greater quantity of tissue to be removed with respect to the instruments currently used.

These objectives according to the present invention are achieved by providing a device for the sampling of tissues in biopsy procedures as specified in claim 1.

Further characteristics of the invention are indicated in the subsequent claims.

The characteristics and advantages of a device for the sampling of tissues in biopsy procedures according to the present invention will appear more evident from the following illustrative and non-limiting description, referring to the enclosed schematic drawings, in which:

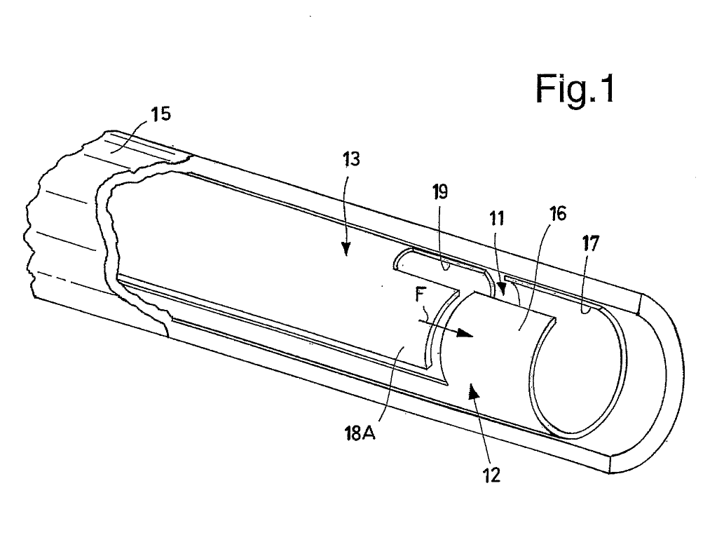

FIG. 1 is a perspective cross-section of an embodiment of a device for the sampling of tissues in biopsy procedures according to the invention in a first “rest” position;

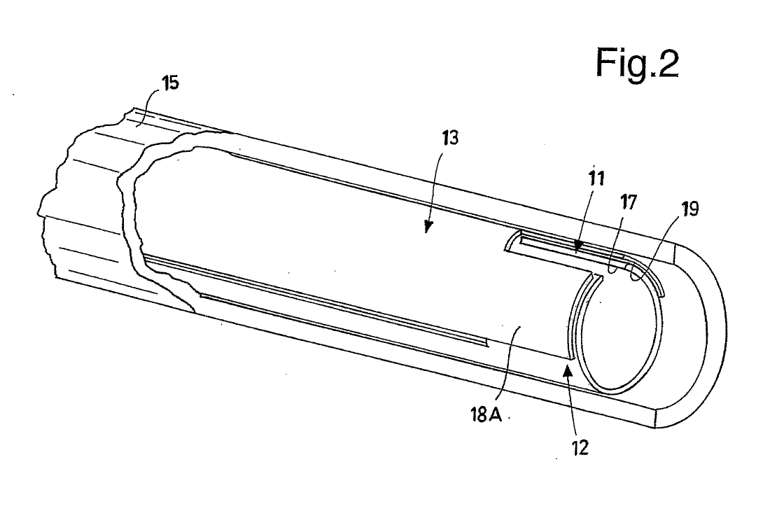

FIG. 2 is a perspective cross-section of the device for the sampling of tissues in biopsy procedures of FIG. 1 in a second operating position;

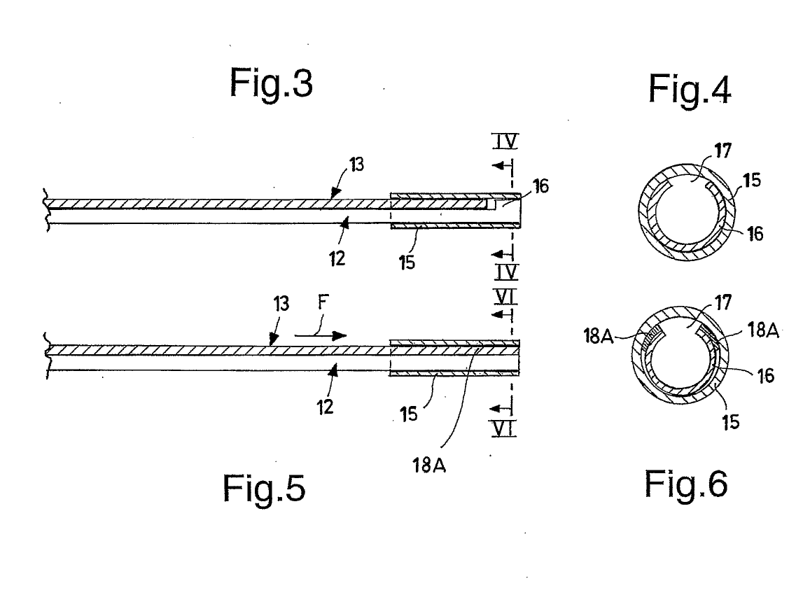

FIG. 3 is a vertical sectional view of the device of FIG. 1;

FIG. 4 is a sectional view of the device of FIG. 3 along the line IV-IV;

FIG. 5 is a vertical sectional view of the device of FIG. 2;

FIG. 6 is a sectional view of the device of FIG. 5 along the line VI-VI;

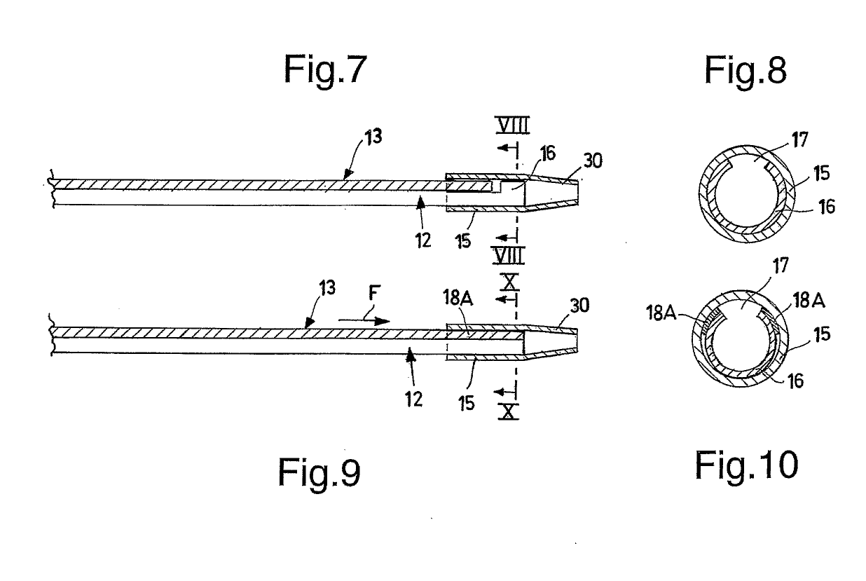

FIG. 7 is a vertical sectional view of a device for the sampling of tissues in biopsy procedures according to the invention in a first “rest” position;

FIG. 8 is a sectional view of the device of FIG. 7 along the line VIII-VIII;

FIG. 9 is a vertical sectional view of the device of FIG. 7 in a second operating position;

FIG. 10 is a sectional view of the device of FIG. 9 along the line X-X;

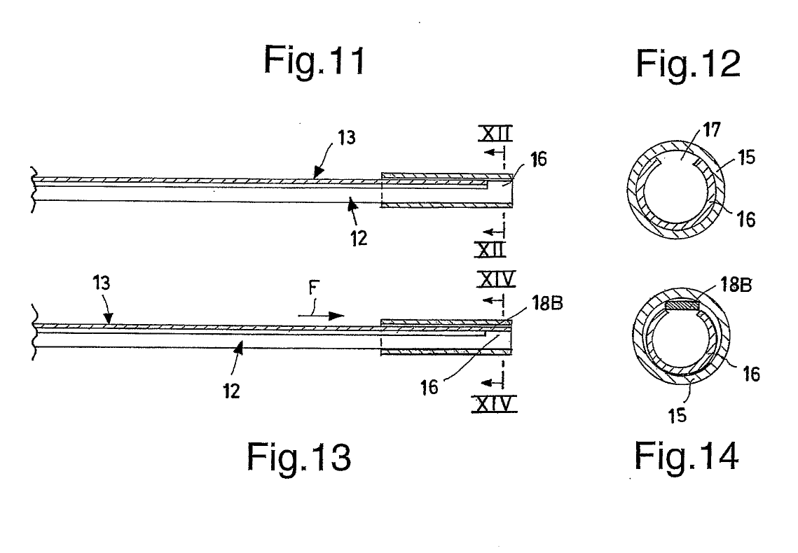

FIG. 11 is a vertical sectional view of a device for the sampling of tissues in biopsy procedures according to the invention in a first “rest” position;

FIG. 12 is a sectional view of the device of FIG. 11 along the line XII-XII;

FIG. 13 is a vertical sectional view of the device of FIG. 11 in a second operating position;

FIG. 14 is a sectional view of the device of FIG. 13 along the line XIV-XIV;

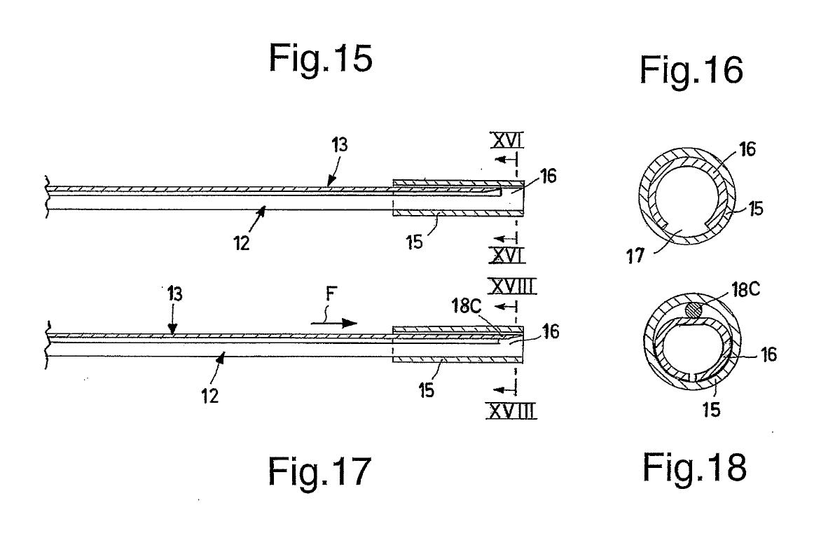

FIG. 15 is a vertical sectional view of a device for the sampling of tissues in biopsy procedures according to the invention in a first “rest” position;

FIG. 16 is a sectional view of the device of FIG. 15 along the line XVI-XVI;

FIG. 17 is a vertical sectional view of the device of FIG. 15 in a second operating position;

FIG. 18 is a sectional view of the device of FIG. 17 along the line XVIII-XVIII;

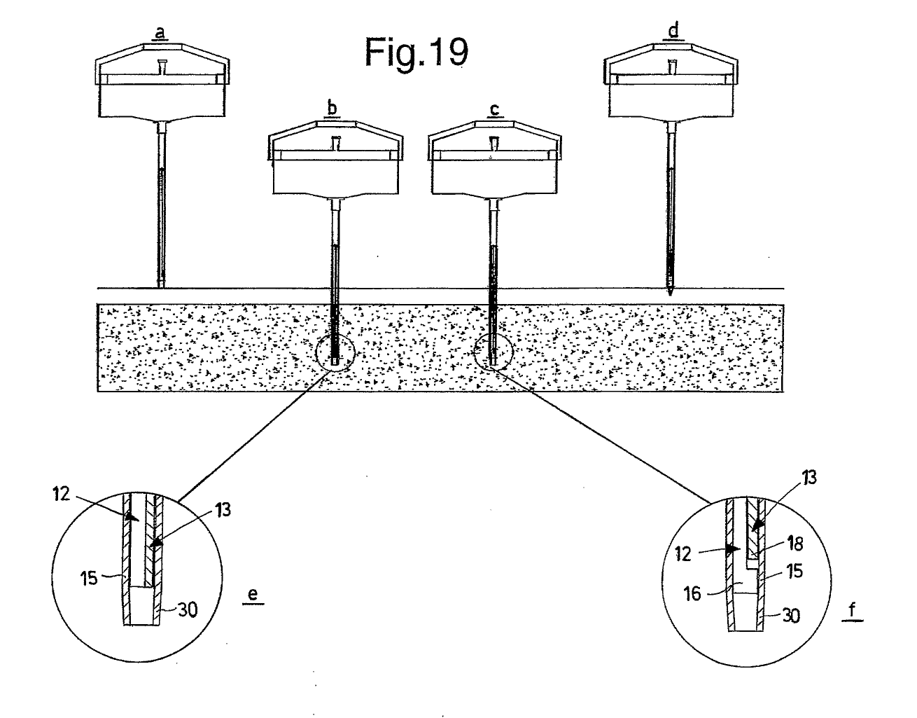

FIG. 19a-19d is a schematic side view of the phases of use of a device for the sampling of tissues in biopsy procedures according to the invention;

FIG. 19e is an enlarged sectional view of a detail shown in 19b;

FIG. 19f is an enlarged sectional view of a detail shown in 19c;

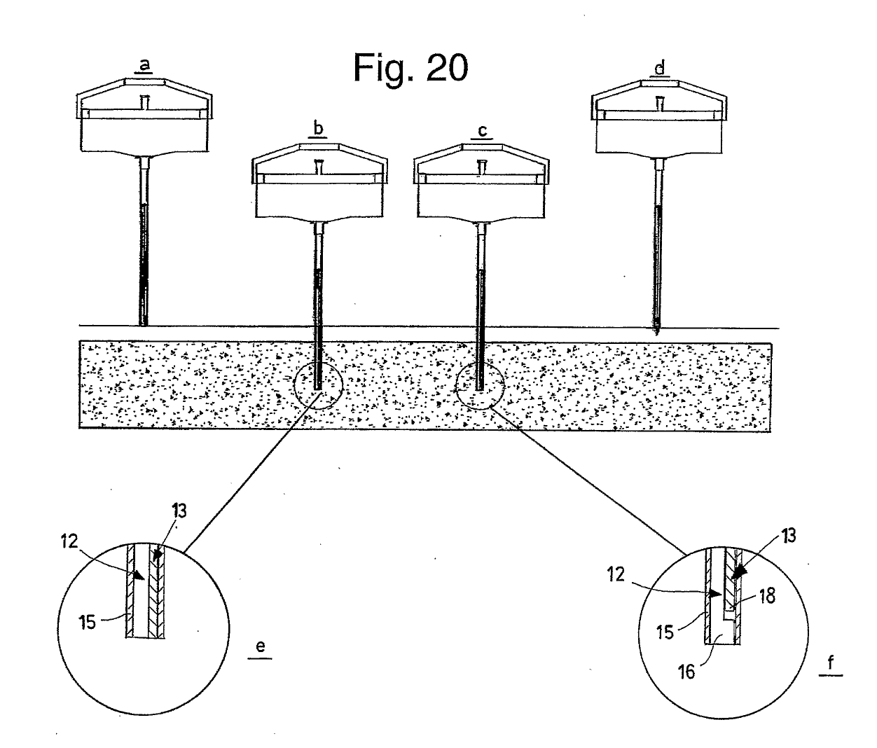

FIG. 20a-20d is a schematic side view of the phases of use of another device for the sampling of tissues in biopsy procedures according to the invention;

FIG. 20e is an enlarged sectional view of a detail shown in 20b;

FIG. 20f is an enlarged sectional view of a detail shown in 20c.

With reference to the figures, these show a device for the sampling of rigid and soft, human and animal tissues in biopsy procedures, indicated as a whole with 10.

Said device 10 for the sampling of tissues in biopsy procedures comprises a withdrawal group 11 situated inside a cannula 15.

As shown in the examples in the figures, said cannula 15 is generally in cylindrical form.

The withdrawal group 11 consists of a withdrawal element 12 provided with an end 16 and a sliding element 13, along a direction indicated in the figures with the arrow F, with respect to both the cannula 15 and the withdrawal element 12.

Said sliding element 13 is provided with an end 18 which can be selectively moved by an operator from a first position not overlying said end 16 and a second position interposed between said cannula 15 and said end 16.

The device 10 can envisage the use of a cannula 15 and a withdrawal group 11 having various geometries. According to preferred embodiments, shown in the figures, the cannula 15 has a cylindrical form, possibly with a tapered end as in FIGS. 7 and 9, and the end 16 is cylindrical, possibly equipped with a split along a generatrix creating at least one opening 17.

According to a preferred embodiment, shown in FIGS. 1-9, the device 10 shows a sliding element 13 and a withdrawal element 12 which can be longitudinally coupled to form a cylindrically-shaped element coaxial with respect to the cannula 15. During the non-use phases of the device 10, the sliding element 13 not only is not superimposed with respect to the cylindrical element 16, which can have at least one opening 17, but is never superimposed with respect to the whole withdrawal element 12, exactly forming, as a complement thereto, the above-mentioned cylindrically shaped element.

According to this illustrative embodiment, only in the above second position, which is applied during the operating phases of the device 10, is the end 18A of the sliding element 13 superimposed with respect to the split cylindrical element 16, FIGS. 6, 10, 14, 18.

The device 10 according to the invention described above, can have an end 18A which in the operating phase is superimposed with respect to the split cylindrical end 16 in correspondence with at least one opening 17.

In this case, shown as an example in FIGS. 1, 2, 6 and 10, the end 18A also advantageously has a split portion, or opening 19, for not hindering and/or allowing the portion of tissue contained inside the split cylindrical opening 16 to exit during the reciprocal sliding.

According to an embodiment (not shown), the split cylindrical end 16 can also have two openings arranged, for example, diametrically opposite each other.

In this case, the end 18A also has openings corresponding to those present in the split cylindrical end 16 for not hindering and/or allowing the portion of tissue contained inside the split cylindrical opening 16 to exit during the reciprocal sliding.

In another embodiment, the device 10 can have an end 18 which, in the operating phase, is superimposed with respect to the split cylindrical end 16 not in correspondence with the at least one opening 17.

In this case, the absence of openings may not be envisaged on the end 18 without jeopardizing the good functioning of the device 10.

In order to facilitate the sliding of the end 18 on the split cylindrical end 16, especially in embodiments in which the two elements in the first rest position are never superimposed along the whole of their longitudinal development, seats can be envisaged, such as suitably smoothed profiles.

In other embodiments according to the present invention, the end 18 of the sliding element 13 can have a flat form 18B, FIGS. 11-14, or a full wire form 18C, FIGS. 15-18.

The same effect as the previous embodiments described i.e. compressing the end 16, is also obtained with these geometries during the second position.

The device 10 according to the present invention can also comprise a cannula 15 which has a tapered conical distal point 30.

The withdrawal group 11, consisting of the withdrawal element 12 and the sliding element 13 can be advantageously completely extracted from the cylindrical cannula 15, to facilitate the removal of the tissue imprisoned therein, and said group 11 can also be reused after subsequent reinsertion either in the same or in a cannula 15 similar to that of the previous use.

Finally, a sharpened full wire can also be envisaged to be inserted in the distal point for allowing greater penetration in the tissues.

It is extremely easy to understand the functioning of the device, object of the invention.

The device 10 is initially allowed to penetrate through a particular tissue to be analyzed, for example bone tissue, FIGS. 19a-b or 20a-b. Once it has reached the tissue, a part of the same is situated inside the cavity of the cannula 15. The sampling operation begins with this arrangement.

This, represented in FIG. 19b or 20b and 19e or 20e, can be defined as a “rest” position as although the tissue is inside the device 10, it is not withheld, should the device be withdrawn without effecting other operations.

The device 10 passes from this rest position to a second “operating” position, represented in FIG. 19c or 20c and 19f or 20f, when the advancing of the end 18 of the sliding element 13 on the split cylindrical end 16 of the withdrawal element 12 is commanded selectively or by an operator.

In this position, the above device on the split cylindrical end 16 is in a conformation which has greater compression with respect to the previous “rest” position in which no element was interposed between it and the cannula 15.

As a result of this overlapping a narrowing is caused of the internal diameter on the split cylindrical end 16 around the tissue.

This narrowing creates a clamping position of the device on the sample of tissue which allows the doctor to extract the needle from the patient and capture the tissue.

This operation takes place without having to effect painful luxation operations, as, thanks to the narrowing, the fragment of tissue is gripped and detached from the underlying tissue without effecting flexures or rotations of the same, FIG. 19d or 20d.

Furthermore, thanks to said device 10, the operator does not have to insert a further device into the instrument during the sampling operation which allows a greater quantity of tissue to be removed with respect to the instruments currently used.

It can thus be seen that a device for the sampling of tissues in biopsy procedures according to the present invention achieves the purposes specified above.

The device for the sampling of tissues in biopsy procedures of the present invention thus conceived can undergo numerous modifications and variants, all included in the same inventive concept.

Furthermore, in practice, the materials used, as also the dimensions and components, can vary according to technical requirements.

1. A device (10) for the sampling of tissues in biopsy-procedures comprising a sampling group (11) situated inside a cannula (15), wherein said withdrawal group (11) consists of a withdrawal element (12) provided with an end (16) and a sliding element (13) with an end (18, 18A, 18B, 18C) which can be moved from a first position not overlying said end (16) and a second position interposed between said cannula (15) and said end (16).

2. The device (10) according to claim 1, characterized in that said cannula (15) is cylindrical and said end (16) is cylindrical and split along a generatrix creating at least one opening (17).

3. The device (10) according to claim 2, characterized in that said end is flat (18B).

4. The device (10) according to claim 2, characterized in that said end has a full round wire geometry (18C).

5. The device (10) according to claim 2, characterized in that said withdrawal element (12) and said sliding element ‘ (13) can be longitudinally coupled to form an element having a cylindrical form coaxial to said cannula (15) wherein in said first position, said sliding element (13) is never superimposed with respect to said withdrawal element (12) and in said second position, only said end (18A) is superimposed with respect to said split cylindrical end (16).

6. The device (10) according to claim 5, characterized in that said end (18A) and said split cylindrical end (16) have seats for facilitating the reciprocal overlapping in said second position.

7. The device (10) according to claim 2, characterized in that in said second position, said end (18A) is superimposed with respect to said split cylindrical end (16) not in correspondence with said at least one opening (17).

8. The device (10) according to claim 2, characterized in that in said second position, said end (18A) is superimposed with respect to said split cylindrical end (16) in correspondence with said at least one opening (17).

9. The device (10) according to claim 8, characterized in that said end (18A) which is superimposed with respect to said split cylindrical end (16) in correspondence with said at least one opening (17) also has at least one opening (19) which is superimposed with respect to said at least one opening (17) without reducing its passage.

10. The device (10) according to claim 1, characterized in that said cannula (15) can be equipped with a tapered conical distal point (30).

11. The device (10) according to claim 1, characterized in that it comprises a sharpened full wire in the distal point which is inserted in said withdrawal group (11) for allowing penetration into the tissues.

12. The device (10) according to claim 1, characterized in that said withdrawal group (11) can be extracted and reinserted into said cannula (15).
